(19) United States
(12) Patent Application Publication
(10) Pub. No.: US 2011/0008854 A1
(43) Pub. Date: Jan. 13, 2011

(54) PROCESS FOR THE GENERATION OF ALGAL OIL AND ELECTRICITY FROM HUMAN AND ANIMAL WASTE, AND OTHER HYDROCARBON SOURCES

(76) Inventor: Andrew G. Mitchell, Chardon, OH (US)

Correspondence Address: Emerson, Thomson & Bennett, LLC, 1914 Akron-Peninsula Road, Akron, OH, 44313, US

(21) Appl. No.: 12/832,697

(22) Filed: Jul. 8, 2010

Related U.S. Application Data
(63) Continuation-in-part of application No. 12/692,038, filed on Jan. 22, 2010.

(30) Foreign Application Priority Data
Jul. 8, 2009 (ZA) 2009/04785
Dec. 17, 2009 (ZA) 2009/08980

(51) Int. Cl. C12P 7/64; C02F 3/34; C12M 1/00

(52) U.S. Cl. 435/134; 435/262; 435/290.1

(57) ABSTRACT

A method for the generation of electricity includes producing a paste from hydrocarbon waste, delivering the paste to a pyrolysis unit, rendering the paste into a gaseous product stream, allowing the stream to flow through a suitable filter device, extracting energy from the stream, producing electricity, converting water to hydrogen via a water gas shift reaction, allowing the reacted stream to flow to a reciprocating compressor, allowing any remaining stream to pass through compression, cooling, condensing, and storing carbon dioxide in a high pressure tank, allowing any remaining stream to be split, one part being combusted in a gas turbine producing electricity and the other part being recycled to the plasmolysis unit, injecting exhaust gas into a feed stream which flows to a degasification chamber, and allowing carbon dioxide and residual water from a storage tank to be expanded, passed through the heat exchanger and injected into the aquaculture feed.

This application claims priority to South African Patent No. 2009/08980, filed Dec. 17, 2009 and South African Patent No. 2009/04785, filed Jul. 8, 2009, the contents of which are incorporated by reference. This application also claims priority to, and is a continuation-in-part of, U.S. Ser. No. 12/692,038, filed Jan. 22, 2010, entitled PLASTIC DISPOSABLE REACTOR SYSTEM, which is incorporated herein by reference. The invention is a process that facilitates the generation of hydrogen, biodiesel and carbon from algae, human and animal waste, and other hydrocarbon sources. The process facilitates the capture and recycling of all the carbon dioxide produced via algal aquaculture.

### FIELD

### Background

There is a growing worldwide demand for a transformation in the production of energy, from fossil fuels to renewable resources and for a concurrent reduction in the emission of pollutants. Different enabling technologies such as wind power, geothermal, hydroelectric, solar, tidal, and various agricultural technologies have been developed to harness energy with varying degrees of success and different strategies have been developed to curb pollutants; however a commercially viable and intrinsically safe method has yet to be developed. This invention describes a process that has the potential to deliver renewable energy with miniscule pollutant emissions.

The use of animal and human waste for the generation of useful energy has been investigated extensively and is currently pursued as a commercial activity in many countries of the world. Various patents have been issued for processes that enable these objectives. Nielsen et al (US Patent 2009/0064581 A1) for example recently published a paper describing the use of plasma assisted destruction of municipal waste stream reaction residues. The gasification process (via various combustion methods including plasma torch) is currently employed in commercial applications particularly in Japan, where landfill disposal of waste is expensive due to the limited availability of space. The gasification process produces a synthesis gas containing carbon monoxide, carbon dioxide, water, and hydrogen. The synthesis gas is often combusted in combined heat and power (CHP) plants.

Removal of hydrogen from hydrogen rich streams has been widely reported using a variety of mechanisms, the most prevalent being Pressure Swing Adsorption (PSA) and more recently the use of membrane technology. PSA systems are not energy efficient however due to the high inlet pressure requirement. Membrane separation techniques employ a variety of membrane materials amongst which palladium or palladium alloys are the most prevalent. Some, as in Munschau et al (US Patent 2008/0000350 A1) incorporate the simultaneous removal and production of hydrogen via the water gas shift reaction (WGS), via:

CO+H₂O [symbol] CO₂+H₂ Equation 1:Water Gas Shift Reaction

Munschau et al have reported an invention that incorporates the use of a catalyst deposited on the outer surface of the membrane to promote the water gas shift reaction and simultaneously allow the adsorption and removal of the hydrogen on the membrane surface. As the catalyst is poisoned by small amounts of sulphur, the use of these membranes is typically restricted to streams with sulphur concentrations of less than 20 ppm.

The sequestration of carbon has received much recent attention and papers discussing algae aquaculture as a viable method have been published extensively. So has the treatment of wastewater using aerobic and anaerobic photobioreactors. Patents and other papers on both topics have been summarized by Elefritz et al (U.S. Pat. No. 7,455,765).

Lewnard et al (US Patent Appln. No. 2008/0178739) provide a comprehensive review of both open and closed system designs, as well as a hybrid method for cultivating algae in large closed spaces. The main issues cited by most authors are the propensity for contamination in open systems as well as a fairly low yield in terms of algal growth per unit land area compared to closed systems which have the comparative high capital cost per unit of land area. Closed systems have the advantage of increased carbon dioxide availability. Freeman (US Patent Appln. No. 2008/0254529) describes a process whereby liquid culture mediums are exposed to closed carbon dioxide/air mixtures. Whitton (US Patent Appln. No. 2008/0286851) describes a flexible integrated closed system constructed of thin plastics which can potentially be folded up and transported to different sites or mounted on earthen bearms. The inclusion of gas spargers is discussed. Howard et at (US Patent 2008/0299643) discloses a variant on the hybrid open/closed system with plastic pond covers and the introduction of diffused CO₂.

### SUMMARY

In the process described, moist waste solids are delivered to a pyrolysis unit (pyrolysis is the chemical decomposition of a condensed substance by heating) employing one or more gas plasmolysis torches (plasmolysis is the chemical decomposition of matter employing high temperature gas plasma). The moist solids have been macerated to a suitable particle size fraction and, in the moist condition, constitute a paste. The paste is introduced into the pyrolysis chamber through concentric cylinders (the “waste feed former”) forming a paste cylinder with an internal diameter greater than that of the plasmolysis torch external diameter. A plasmolysis torch is situated inside the paste cylinder at a sufficient height above the waste former so as not to cause any thermal damage of the equipment. Secondary torches are placed outside of the paste cylinder such that the combined effect of the plasmolysis torches completely renders the waste into a gaseous product stream. Other gas inlet nozzles allow gas into the chamber in sufficient quantities that all suspended solids are entrained. The gases flow through the radiant heat exchanger which conveys energy to superheated steam. The steam drives a steam turbine and is condensed and recycled. Following the radiant heat exchanger, the gases flow through a bag particle filter and into a combined secondary heat exchanger and catalytic converter. In this unit, further energy is extracted from the gases and water is converted to hydrogen via the water gas shift reaction. The reacted gases then flow to a hydrogen separation device in which hydrogen is extracted, compressed and stored in gas cylinders. The remaining gases are circulated to a compressor expander unit where a purge stream flows through the expander providing the energy to compress the recycle stream. The purge stream is delivered to the in line mixers for mixing with the algal aquaculture water feed and the recycle stream is compressed and returned to a gas storage vessel. From the gas storage vessel the recycle stream is fed back to the plasmolysis unit.

In another embodiment of the process described, moist waste solids are delivered to a pyrolysis unit (pyrolysis is the chemical decomposition of a substance by heating) employing one or more gas plasmolysis torches (plasmolysis is the chemical decomposition of matter employing high temperature gas plasma). The moist solids have been macerated to a suitable particle size fraction and, in the moist condition, constitute a paste. The paste is introduced into the pyrolysis chamber through either 1) concentric cylinders (the “waste feed former”) forming a paste cylinder with an internal diameter greater than that of the plasmolysis torch external diameter or 2) through a feed tube in the form of a solid cylinder with two or more plasmolysis torches arranged so that the flames impinge on the cylinder at an acute angle to the axis of the cylinder. In the case of the concentric cylinder feed, a plasmolysis torch is situated inside the paste cylinder at a sufficient height above the waste former so as not to cause any thermal damage of the equipment. Secondary torches are placed outside of the paste cylinder such that the combined effect of the plasmolysis torches completely renders the waste into a gaseous product stream. Other gas inlet nozzles allow gas into the chamber in sufficient quantities that all suspended solids are entrained. The gases flow through the radiant heat exchanger which conveys energy to superheated steam. The steam drives a steam turbine and is condensed and recycled. Following the radiant heat exchanger, the gases flow through a particle filter (which may be a centrifuge or bag filter or other suitable device known in the art) and into a combined secondary heat exchanger and catalytic converter. In this unit, further energy is extracted from the gases and water is converted to hydrogen via the water gas shift reaction. Methane is converted to carbon monoxide and hydrogen via the steam reformer reaction:

CH₄+H₂O [symbol] CO+3H₂ Equation 2:Steam Reformer Reaction

The reacted gases flow to a condensing heat exchanger wherein water is condensed and removed. The remaining gases are compressed in a three stage reciprocating compressor with interstage cooling and interstage removal of hydrogen via membrane separation. The interstage removal of hydrogen is incorporated in this invention. The final stage of compression increases the partial pressure of the water vapor and carbon dioxide to such a degree that upon cooling in the post compression heat exchangers the water vapor condenses and with further cooling the carbon dioxide subsequently liquefies. The liquefied carbon dioxide is stored in a high pressure storage tank. The residual gas stream is recycled to the pyrolysis chamber and to the plasmolysis torches. Carbon dioxide from the storage tank is expanded through a heat exchanger (which may be situated in a cold storage chamber) and delivered to the in line mixers for mixing with the algal aquaculture water feed during daytime operation.

The algal aquaculture feed stream is heated by the condensate steam from the process.

In the process described, carbon dioxide containing gas is injected into sufficient water under pressure to dissolve the carbon dioxide using in line mixers. Carbon Dioxide rich water is pumped to a Plastic Disposable Reactor (“PDR”) train, consisting of multiple units of the PDRs. The PDRs have been inoculated with and contain growing algae. The nutrient rich waters are fed upwards at low linear velocities through the PDRs and the resultant oxygen enriched water is drawn through a filter at the top of the PDR. The design of the filtration device and its fixture to the PDR is incorporated in this invention. The water is preheated to between about 24° C. and about 32° C. for optimal algae growth. (This temperature may change for other species of microbes). The internal diameter of the PDR may vary from just greater than 0 to about 5 or more inches but is not limited to this upper limit. The height of the PDR may vary from just greater than 0 to about 24 or more feet but is not limited to this upper limit. The wall thickness of the PDR may vary from just greater than 0 to about ¼ inch or more but is not limited to this upper limit. The thickness of the reactor wall is determined by the design operating pressure, the internal diameter and height of the vessel using typical engineering considerations. The inlet and exit of the PDR may have an internal pipe thread, an external pipe thread, or an external tube connector. This may be Imperial (BSP), metric (ISO), or US National Pipe Thread (NPT) and may be more or less than the typical 1 inch diameter. The design of the PDR and the filtration device is incorporated in the invention. The material of choice for the PDR for the purpose of aquaculture of algae is polyethylene teraphthalate (PET); however the PDR may be made of other suitable materials including, but not limited to, clear polyvinyl chloride (PVC), Polypropylene (PP), polyethylene (PE), high density polyethylene (HDPE), cross linked polyethylene (PEX), clear polycarbonate and other plastics. FIG. 3 shows a plant layout which removes carbon dioxide from an incoming gaseous stream by dissolution in water at ambient or elevated temperature and pressure. The carbon dioxide rich water stream is conveyed through a series of three way ball valves (all valves with the exception of valve 3 which is a flow control valve) to the PDR units. In train 1 the valves are configured to allow the carbon dioxide rich water stream to pass upwards through the PDR train containing algae. The algae in the course of photosynthetic metabolism convert the carbon dioxide to various complex organic molecules and oxygen. The oxygen (dissolved and gaseous) is conveyed from the algae by the continued upward motion of the water. In the second PDR train, the valves are configured such that potable water is fed to the top of the PDR train allowing water and algae to be drawn from the bottom of the train and “harvested.” Once a fraction (in one embodiment, but not limited to, about one-half) of the algae has thus been withdrawn from each PDR, the valves are reconfigured to allow either carbon dioxide enriched water or potable water (depending on the light cycle—i.e. either day or night) up through the PDR. A further embodiment of the described operation allows for the use of a bleaching agent in conjunction with potable water to clean the interior surface of the PDRs. Once this cycle has been completed, the cleaned PDRs will have to be re-inoculated with growing algae. This cleaning is helpful for continued maximum availability of light throughout the PDR. After a period of time has elapsed, wherein the reactors may need to be replaced, the reactors are disconnected from the train and replaced with new reactors. The old reactors may be washed and sent for recycling. The number of PDRs in a train and the number of trains employed for any given site will depend on various factors including, but not limited to, the quantity of gas to be treated, the availability of land space, the size distribution of the PDR units and the climatic conditions where the facility is to be situated. FIG. 4 shows a PDR with the filtration mechanism attached. The design of the PDRs has been discussed in the summary. The filtration device is the counterpart of the female pipe thread—a male threaded fitting. The fitting incorporates a porous filtration medium in the shape of a plug that is affixed to the tube. The bottom of the PDR is affixed to the fluid conveying pipe by means of a suitable sized male threaded connection and flexible hose. FIG. 5 shows a series of connected PDRs forming a “Train.” The trains can be suspended from an external support which attaches to the top water conveying pipe.

### BRIEF DESCRIPTION OF THE DRAWINGS

Further features, benefits and advantages of the invention will become evident from the following description of exemplary embodiments with reference to the drawings, in which:

FIG. 1 shows a schematic of the plasmolysis unit waste feed former;

FIG. 2 shows a process flow;

FIG. 3 shows a process flow diagram for the removal of carbon dioxide from a carbon dioxide rich stream and subsequent treatment of the carbon dioxide saturated or partially saturated water in two trains of PDRs;

FIG. 4 shows a detailed cross section of a PDR;

FIG. 5 shows a schematic of a PDR train; and,

FIG. 6 shows a process flow diagram for the gasification of waste, the generation of electricity and the dissolution of carbon dioxide in water.

### DETAILED DESCRIPTION

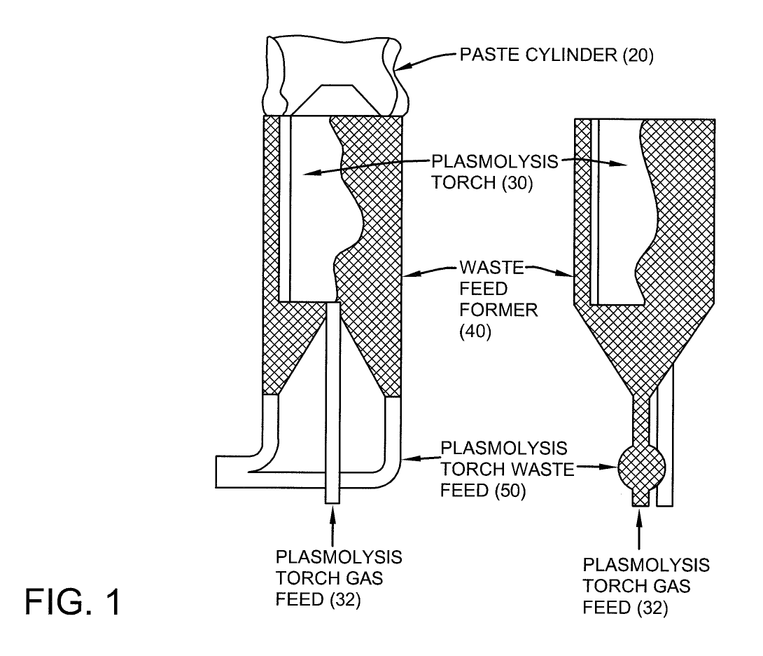

FIG. 1 shows one embodiment of the plasmolysis unit waste feed former (28). The waste feed is introduced from the waste solid maceration tank (22) by the action of a mechanical auger and/or a positive displacement device that allows delivery to the unit at an operating pressure of between 0 to 10 bar (g). The waste is fed through the delivery system to a cylindrical device (20) resulting in a continuously formed cylinder of solid feed that moves upwards at a specified linear velocity. The cylinder (20) surrounds the primary gasification torch (30) and is also fired upon by as many as four secondary plasmolysis torches (30), situated externally to the cylinder (20) and at such impingement angles as to optimally and completely gasify the waste feed cylinder.

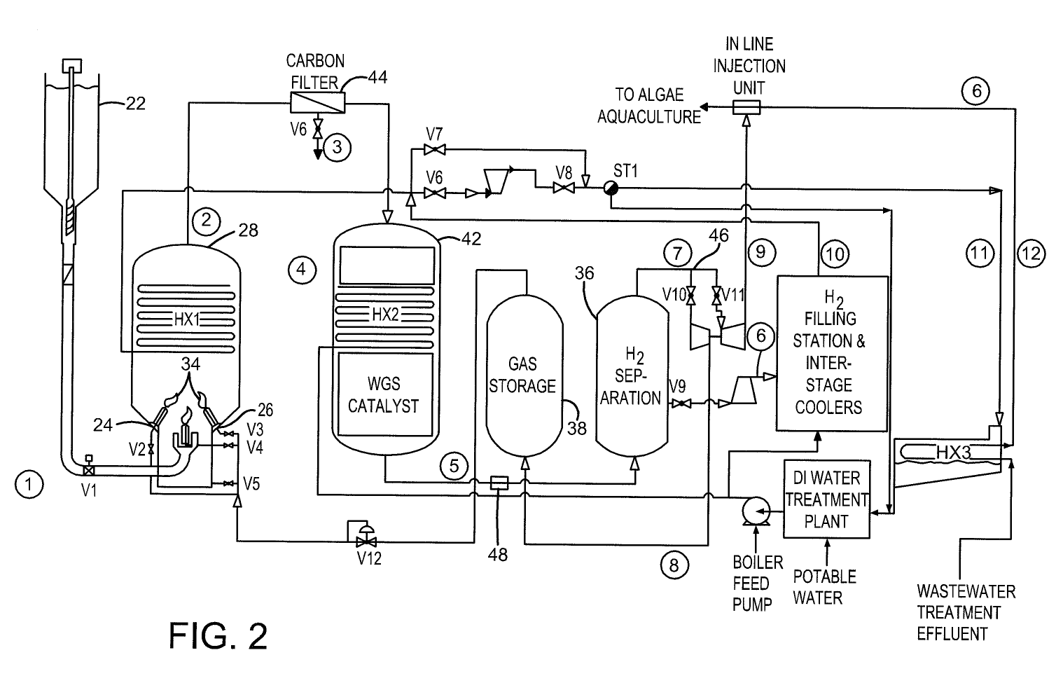

FIG. 2 shows one embodiment of a plant layout which conveys waste from the maceration tank (22) to a plasmolysis combustion furnace described above (stream 1). In the furnace (28), secondary gas inlet nozzles (24, 26) allow sufficient gas to circulate upwards in the gasification unit thereby retaining any suspended solids (carbon and ash) that form in the plasmolysis of the waste and generate a circular flow path for maximum residence time within the radiant heat exchanger. FIG. 2 shows a process flow diagram for the transport of the moist waste solid feed to the plasmolysis unit, gasification of the waste solid stream, generation of superheated steam in the radiant section of the gasification unit, generation of electricity from the steam, recirculation of condensate steam, convection of the plasmolysis unit exhaust through a bag filter unit (44) to a secondary heat exchanger (HX2) and catalytic converter (48), to a hydrogen extraction device (36), to an expander/compressor unit (46) wherein the stream is split into a purge stream (9) which is expanded and feeds an algal aquaculture unit and a recycle stream (8) which delivers compressed exhaust gas to a storage vessel (38) and is returned to the pyrolysis unit. The purge stream is injected into the algal aquaculture feed stream which is delivered to the algae generation facility.

Combustion exhaust gases (stream 2) are extracted from the radiant heat exchange section (HX1) of the furnace (28), forced through a high temperature bag dust filter (44) which removes suspended solids (predominantly carbon in various stages of activation—stream 3) and then through a convective heat exchanger (42). (Boiler water—stream 4—is partially vaporized in the convective heater (42) and superheated in the radiant heat exchanger (HX2) and delivered to the steam turbine for production of electricity). The convective heat exchanger (42) may contain a solid catalyst which promotes the water gas shift reaction defined above. Cooled reacted gas (stream 5) then flows through a catalytic converter (48) which drives the water gas shift reaction further. Following the catalytic converter (48), the gas flows to a membrane hydrogen extraction device (36) which delivers purified hydrogen to a compressor unit for storage in cylinders (stream 6). The use of steam produced by the interstage cooling of the hydrogen for electricity production is incorporated in this patent application. From there the hydrogen deficit exhaust gas stream (stream 7) is split into a recycle stream (stream 8) and a purge stream (stream 9). The purge stream is expanded and delivered to the algal aquaculture feedwater stream (stream 12) while the recycle stream is compressed and delivered to a gas storage tank (38) from which it is fed back to the plasmolysis combustion furnace (28). The use of the expander/compressor unit for this purpose in the application described is incorporated in the patent application. Waste heat from the hydrogen compressor interstage coolers (stream 10) is used to generate steam for electricity production. Condensing steam (stream 11) is used to heat the algal aquaculture feedwater (stream 12) in the condensate heat exchanger (HX3) prior to the carbon dioxide injection. The use of the condensing steam to heat the algal aquaculture feedwater is incorporated in this patent application. The carbon dioxide enriched water is delivered to the algae generation facility. Water from the condensate heat exchanger is delivered to the boiler water treatment plant.

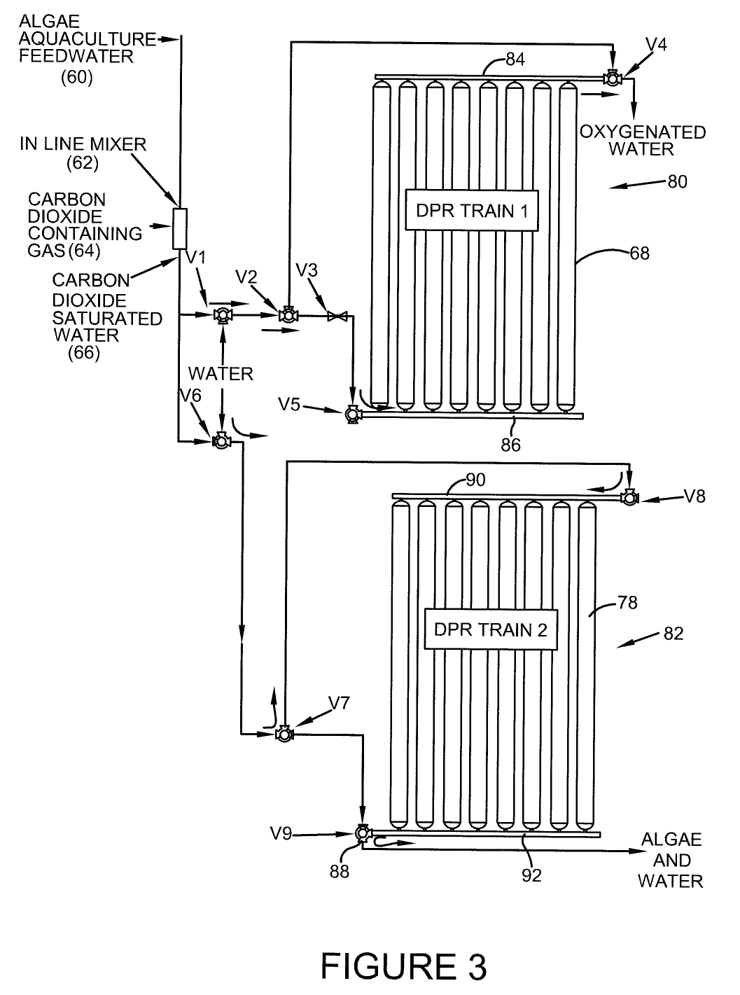

FIG. 3 shows at least one embodiment of a plant layout which removes carbon dioxide from an incoming gaseous stream by dissolution in water at ambient or elevated temperature and pressure. The carbon dioxide rich water stream (66) is conveyed through a series of three way ball valves V1, V2, V4, V5, V6, V7, V8, V9 (all valves with the exception of valve V3 which is a flow control valve) to the PDR (“plastic disposable reactor”—DPR for “disposable plastic reactor” and PDR will be used interchangeably) units (68, 78). FIG. 3 shows the first PDR train (80), having a top fluid conveying pipe (84), bottom fluid conveying pipe (86), algae and water outlet (88), and PDRs (68). It also shows the second PDR train (82), having a top fluid conveying pipe (90), bottom fluid conveying pipe (92), and PDRs (78). In train (80) the valves V1, V2, V3, V5 are configured to allow the carbon dioxide rich water stream to pass upwards through the PDR train (80) containing algae. The algae in the course of photosynthetic metabolism convert the carbon dioxide to various complex organic molecules and oxygen. The oxygen (dissolved and gaseous) is conveyed from the algae by the continued upward motion of the water. In the second PDR train (82), the valves V6, V7, V9 are configured such that potable water is fed to the top of the PDR train allowing water and algae to be drawn from the bottom fluid conveying pipe (92) of the train and “harvested.” Once a fraction (in one embodiment, but not limited to, about one-half) of the algae has thus been withdrawn from each PDR (68, 78), the valves are reconfigured to allow either carbon dioxide enriched water or potable water (depending on the light cycle—i.e. either day or night) up through the PDR (68, 78).

Carbon dioxide rich water is pumped to the PDR train (80, 82), consisting of multiple PDRs (68, 78). The PDRs have been inoculated with and contain growing algae. The nutrient rich waters are fed upwards at low linear velocities through the PDRs and the resultant oxygen enriched water is drawn through a filter at the top of the PDR. The design of the filtration device and its fixture to the PDR is incorporated in this invention.

The water is preheated to between about 24° C. and about 32° C. for optimal algae growth. (This temperature may change for other species of microbes). The internal diameter of the PDR may vary from just greater than 0 to about 5 or more inches but is not limited to this upper limit. The height of the PDR may vary from just greater than 0 to about 24 or more feet but is not limited to this upper limit. The wall thickness of the PDR may vary from just greater than 0 to about ¼ inch or more but is not limited to this upper limit. The thickness of the reactor wall is determined by the design operating pressure, the internal diameter and height of the vessel using typical engineering considerations. The inlet (52) and exit (54) of the PDR (56) may have an internal pipe thread (72), an external pipe thread (70), or an external tube connector (76). This may be Imperial (BSP), metric (ISO), or US National Pipe Thread (NPT) and may be more or less than the typical 1 inch diameter. The material of choice for the PDR for the purpose of aquaculture of algae is polyethylene teraphthalate (PET); however the PDR may be made of other suitable materials including, but not limited to, clear polyvinyl chloride (PVC), Polypropylene (PP), polyethylene (PE), high density polyethylene (HDPE), cross linked polyethylene (PEX), clear polycarbonate and other plastics.

A further embodiment of the described operation allows for the use of a bleaching agent in conjunction with potable water to clean the interior surface of the PDRs. Once this cycle has been completed, the cleaned PDRs will have to be re-inoculated with growing algae. This cleaning is helpful for continued maximum availability of light throughout the PDR.

After a period of time has elapsed, wherein the reactors may need to be replaced, the reactors are disconnected from the train and replaced with new reactors. The old reactors may be washed and sent for recycling.

The number of PDRs in a train and the number of trains employed for any given site will depend on various factors including, but not limited to, the quantity of gas to be treated, the availability of land space, the size distribution of the PDR units and the climatic conditions where the facility is to be situated.

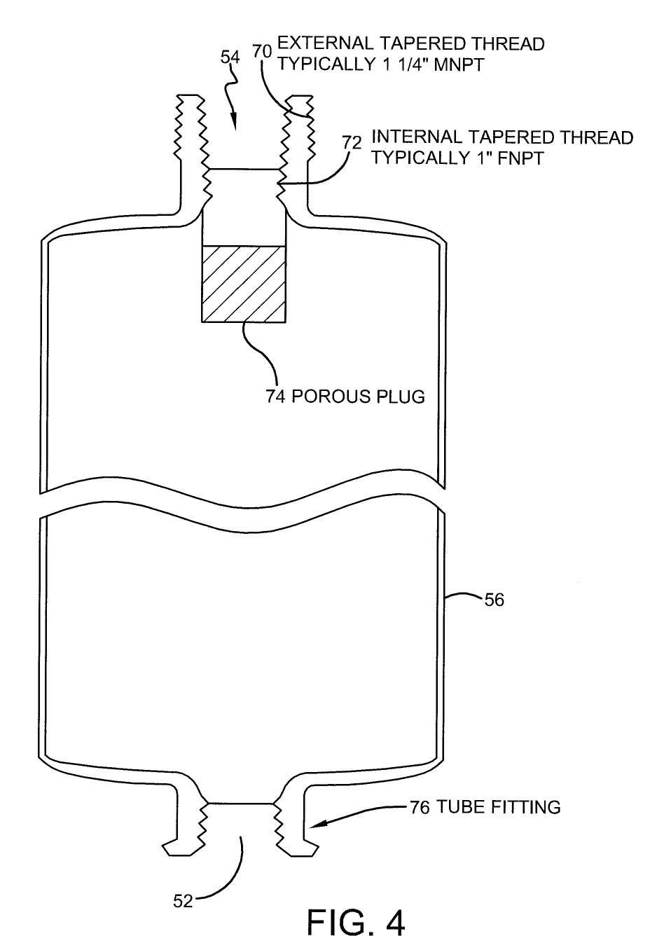

FIG. 4 shows one embodiment of a PDR (56) with the filtration mechanism (74) attached. The design of the PDRs has been discussed in the summary. The filtration device (74) is the counterpart of the female pipe thread—a male threaded fitting. The fitting incorporates a porous filtration medium (74) in the shape of a plug that is affixed to the tube. The bottom of the PDR (56) is affixed to the fluid conveying pipe (86, 92) by means of a suitable sized male threaded connection (76) and flexible hose.

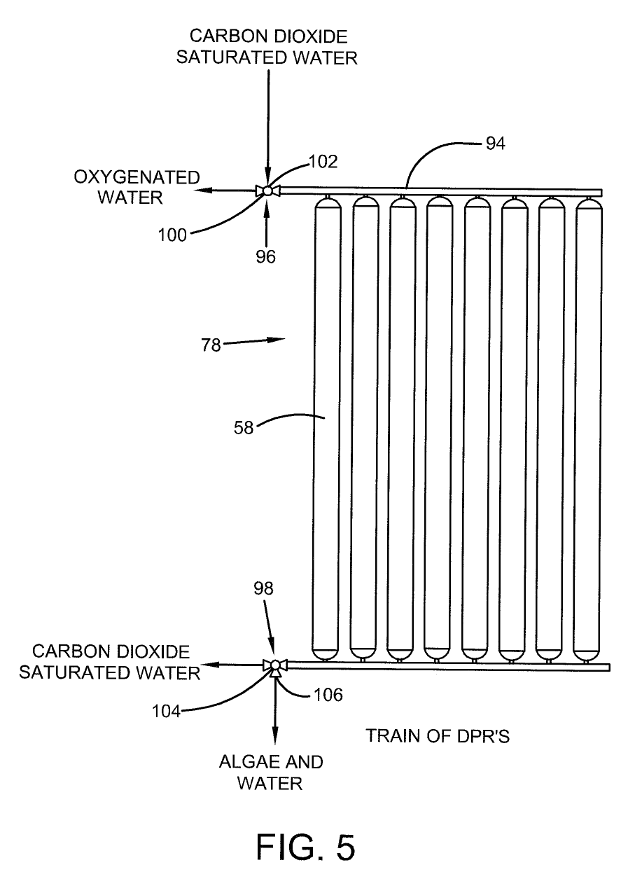

FIG. 5 shows one embodiment of a series of connected PDRs (58) forming a train (78). In the embodiment, these trains (78) will be suspended from an external support which attaches to the top water conveying pipe (94). FIG. 5 also shows valves (96, 98), oxygenated water output (100), carbon dioxide saturated water inlet (102), bottom carbon dioxide saturated water inlet (104), and algae and water outlet (106).

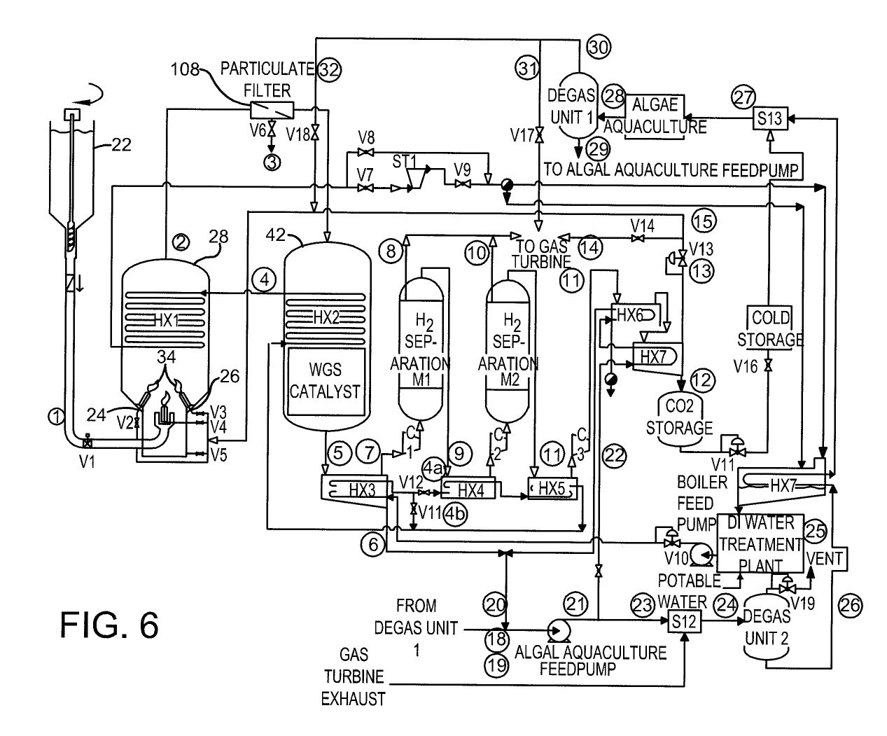

FIG. 6 shows another embodiment of a plant layout which conveys waste from the maceration tank (22) to a plasmolysis combustion furnace (28). In the furnace (28), secondary gas inlet nozzles (24,26) allow sufficient gas to circulate upwards in the gasification unit (28) thereby retaining any suspended solids (carbon and ash) that form in the plasmolysis of the waste and generate a circular flow path for maximum residence time within the radiant heat exchanger (HX1). FIG. 6 shows a process flow diagram for the transport of the moist waste solid feed to plasmolysis unit, gasification of the waste solid stream, generation of superheated steam in the radiant section of the gasification unit, generation of electricity from the steam, recirculation of condensate steam, convection of the plasmolysis unit exhaust through a suitable device to a secondary heat exchanger (HX2) and catalytic converter, to a three stage reciprocating compressor with interstage hydrogen extraction and cooling, to condensing heat exchangers where water condenses, the residual gases are subsequently cooled and carbon dioxide condenses and is stored in a high pressure tank, the residual gases from the carbon dioxide condenser are split into two streams—one being a gas turbine fuel feed and the other a recycle stream to the plasmolysis unit. Gas turbine exhaust is injected into the algae aquaculture feed stream which flows into a degassing chamber, releasing entrained gases (nitrogen and oxygen) to atmosphere. Carbon dioxide and residual water from the high pressure carbon dioxide storage tank are passed through an expansion valve and a heat exchanger (which may be enclosed by a cold storage unit) and injected into the algae aquaculture feed stream. The operating pressure of the algae aquaculture feed stream and algae aquaculture unit may be 1 or more bar absolute.

Hydrogen generated by the process as well as the gas turbine fuel feed are combined and combusted in the gas turbine unit to generate electricity.

Algae extracted from the algae aquaculture unit is dewatered and pressed to extract algae oil (“algal oil”) which may be used in a variety of processes including conversion to biodiesel using conventional methods known in the art. The pressed algae solids may be returned to the waste macerator for reprocessing or used for other purposes.

The above examples have been depicted solely for the purpose of exemplification and are not intended to restrict the scope or embodiments of the invention. The invention is further illustrated with reference to the claims that follow thereto.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The invention has been described with reference to several embodiments. Obviously, modifications and alterations will occur to others upon a reading and understanding of the specification. It is intended by applicant to include all such modifications and alterations insofar as they come within the scope of the appended claims or the equivalents thereof.

Having thus described the invention, it is now claimed:

1. A method for the generation of carbon, hydrogen and carbon dioxide from moist solid wastes, the method comprising the steps of:
macerating the associated solid waste to a paste;
delivering associated moist solid waste to a pyrolysis unit, the pyrolysis unit having at least one gas plasmolysis torch, wherein the waste is delivered into the pyrolysis unit through concentric cylinders forming a paste cylinder with an internal diameter greater than that of a plasmolysis torch external diameter, wherein at least a second torch is located outside of the paste cylinder such that the combined effect of the plasmolysis torches completely renders the waste into a gaseous product stream;
allowing gas into the unit, via at least one gas inlet nozzle, in sufficient quantities that all suspended solids are entrained, wherein the gas flows through a radiant heat exchanger which conveys energy to superheated steam, wherein the steam drives a steam turbine and is condensed and recycled;
following the radiant heat exchanger, the gaseous product stream flows through a bag particle filter and into a combined secondary heat exchanger and catalytic converter, wherein further energy is extracted from the gaseous product stream and water is converted to hydrogen via a water gas shift reaction;
the reacted stream then flows to a hydrogen separation device in which hydrogen is extracted, compressed, and stored in at least one gas cylinder;
any remaining gaseous stream is circulated to a compressor expander unit where a purge stream flows through the expander providing energy to compress recycled stream;
delivering the purge stream to in line mixers for mixing with algal aquaculture water feed;
compressing the recycled stream and returning the recycled stream to a gas storage vessel; and,
feeding the recycled stream back to the plasmolysis unit.

2. A method for the generation of carbon, hydrogen and carbon dioxide from moist solid wastes, the method comprising the steps of:
delivering associated moist solid waste to a pyrolysis unit, the pyrolysis unit having at least one gas plasmolysis torch, wherein the waste is delivered into the pyrolysis unit through concentric cylinders forming a paste cylinder with an internal diameter greater than that of a plasmolysis torch external diameter, wherein at least one plasmolysis torch renders the waste into a gaseous product stream;
allowing the gaseous product stream to flow through a bag particle filter and into a combined secondary heat exchanger and catalytic converter, wherein energy is extracted from the gaseous product stream and water is converted to hydrogen via a water gas shift reaction;
the reacted stream then flows to a hydrogen separation device in which hydrogen is extracted, compressed, and stored in at least one gas cylinder; and,
delivering a purge stream to in line mixers for mixing with algal aquaculture water feed.

3. The method of claim 2, wherein the method further comprises the step of:
allowing gas into the unit, via at least one gas inlet nozzle, in sufficient quantities that all suspended solids are entrained, wherein the gas flows through a radiant heat exchanger which conveys energy to superheated steam, wherein the steam drives a steam turbine and is condensed and recycled.

4. The method of claim 2, wherein at least a second torch is located outside of the paste cylinder such that the combined effect of the plasmolysis torches completely renders the waste into a gaseous product stream.

5. The method of claim 2, wherein any remaining gaseous stream is circulated to a compressor expander unit where a purge stream flows through the expander providing energy to compress recycled stream.

6. The method of claim 2, wherein the method further comprises the steps of:
compressing the recycled stream and returning the recycled stream to a gas storage vessel; and,
feeding the recycled stream back to the plasmolysis unit.

7. An apparatus for facilitating the generation of energy from solid waste, the apparatus comprising:
a pyrolysis unit, the pyrolysis unit having at least one gas plasmolysis torch, wherein the pyrolysis unit has concentric cylinders forming a paste cylinder with an internal diameter greater than that of a plasmolysis torch external diameter;
a radiant heat exchanger operatively connected to a steam turbine; and,
a hydrogen separation device.

8. The apparatus of claim 7, wherein the apparatus further comprises:
at least a second torch located outside of the paste cylinder.

9. The apparatus of claim 8, wherein the apparatus further comprises:
at least one gas inlet nozzle.

10. The apparatus of claim 7, wherein the apparatus further comprises:
a bag particle filter operatively connected to the radiant heat exchanger; and,
a combined secondary heat exchanger and catalytic converter operatively connected to the bag particle filer.

11. The apparatus of claim 7, wherein the apparatus further comprises:
a compressor expander unit;
in line mixers; and,
at least one gas storage vessel.

12. A method for the generation of algal oil and electricity, the method comprising the steps of:
combining and macerating moist hydrocarbon wastes into a paste;
delivering the paste to a pyrolysis unit, the pyrolysis unit having at least one gas plasmolysis torch having an external diameter, wherein the paste is delivered into the pyrolysis unit through concentric cylinders forming a paste cylinder with an internal diameter greater than that of the plasmolysis torch external diameter, wherein at least a second torch is located outside of the paste cylinder such that the plasmolysis torches completely renders the paste into a gaseous product stream;
allowing gas into the unit, via at least one gas inlet nozzle, in sufficient quantities that all suspended solids are entrained, wherein the gas flows through a radiant heat exchanger which conveys energy to superheated steam, wherein the steam drives a steam turbine and is condensed and recycled;
allowing the gaseous product stream to flow through a suitable filter device and into a combined secondary heat exchanger and catalytic converter, wherein further energy is extracted from the gaseous product stream and water is converted to hydrogen via a water gas shift reaction;
allowing the reacted stream to flow to a three stage reciprocating compressor with interstage membrane hydrogen extraction following at least a first stage of compression;
allowing any remaining gaseous stream to pass through a third stage of compression, cooling, condensing, and storing the carbon dioxide in a high pressure tank;
allowing any remaining gaseous stream to be split, one part being combusted in a gas turbine and the other part being recycled to the plasmolysis unit;
allowing exhaust from the gas turbine unit to be injected into an algae aquaculture feed stream which flows to a degasification chamber, the released gas being allowed to vent to atmosphere;
allowing carbon dioxide and residual water from a high pressure storage tank to be expanded, passed through the heat exchanger and injected into the algae aquaculture feed stream using in line mixers for mixing with the algal aquaculture water feed; and,
allowing the algal aquaculture feed stream to be sent to an algal aquaculture unit to facilitate the growth of the algae and carbon sequestration.

13. The method of claim 12, wherein the step of delivering the paste to a pyrolysis unit, the pyrolysis unit having at least one gas plasmolysis torch having an external diameter, wherein the paste is delivered into the pyrolysis unit through concentric cylinders forming a paste cylinder with an internal diameter greater than that of the plasmolysis torch external diameter, wherein at least a second torch is located outside of the paste cylinder such that the plasmolysis torches completely renders the paste into a gaseous product stream comprises:
delivering the paste to a pyrolysis unit, the pyrolysis unit having at least one gas plasmolysis torch, wherein the paste is delivered into the pyrolysis unit through a tube forming a solid cylinder of the paste and at least two plasmolysis torches are located outside of the paste cylinder with the flames impinging at an acute angle to the axis of the cylinder such that the combined effect of the plasmolysis torches completely renders the waste into a gaseous product stream.

14. A method for the generation of algal oil and electricity, the method comprising the steps of:
producing a paste from hydrocarbon waste;
delivering the paste to a pyrolysis unit, the pyrolysis unit having at least one gas plasmolysis torch;
rendering the paste into a gaseous product stream;
allowing the gaseous product stream to flow through a suitable filter device;
extracting energy from the gaseous product stream and converting water to hydrogen via a water gas shift reaction;
allowing the reacted stream to flow to a reciprocating compressor;
allowing any remaining gaseous stream to pass through compression, cooling, condensing, and storing carbon dioxide in a high pressure tank;
allowing any remaining gaseous stream to be split, one part being combusted in a gas turbine and the other part being recycled to the plasmolysis unit;
injecting exhaust gas into an algae aquaculture feed stream which flows to a degasification chamber; and,
allowing carbon dioxide and residual water from a high pressure storage tank to be expanded, passed through the heat exchanger and injected into the algae aquaculture feed stream thereby delivering a purge stream to in line mixers for mixing with the algal aquaculture water feed.

15. An apparatus for facilitating the generation of energy from solid waste, the apparatus comprising:
a pyrolysis unit, the pyrolysis unit having at least one gas plasmolysis torch, wherein the pyrolysis unit has concentric cylinders forming a paste cylinder with an internal diameter greater than that of a plasmolysis torch external diameter;
at least one gas inlet nozzle operatively connected to the pyrolysis unit;
a radiant heat exchanger operatively connected to a steam turbine;
a filter device operatively connected to the radiant heat exchanger;
a convective heat exchanger operatively connected to the filter device;
a condensing heat exchanger operatively connected to the convective heat exchanger;
a three stage reciprocating compressor with interstage membrane hydrogen separation and cooling devices operatively connected to the condensing heat exchanger, wherein the condensing heat exchanger is operatively connected to an outlet of the reciprocating compressor; and,
a high pressure storage tank operatively connected to the condensing heat exchanger.

16. An apparatus for facilitating the generation of energy from solid waste, the apparatus comprising:
a pyrolysis unit, the pyrolysis unit having at least two gas plasmolysis torches, wherein the pyrolysis unit has a tube forming a solid paste cylinder and the plasmolysis torches are arranged such that the impingement angle of the flames is acute to the axis of the cylinder and the number of torches is sufficient to completely gasify the solid cylinder;
at least one gas inlet nozzle operatively connected to the pyrolysis unit;
a radiant heat exchanger operatively connected to a steam turbine;
a filter device operatively connected to the radiant heat exchanger;
a convective heat exchanger operatively connected to the filter device;
a condensing heat exchanger operatively connected to the convective heat exchanger;
a three stage reciprocating compressor with interstage membrane hydrogen separation and cooling devices operatively connected to the condensing heat exchanger, wherein the condensing heat exchanger is operatively connected to an outlet of the reciprocating compressor; and,
a high pressure storage tank operatively connected to the condensing heat exchanger.

17. An apparatus for facilitating the generation of energy from solid waste, the apparatus comprising:
a pyrolysis unit, the pyrolysis unit having at least one gas plasmolysis torch;
at least one gas inlet nozzle operatively connected to the pyrolysis unit;
a radiant heat exchanger operatively connected to a steam turbine;
a three stage reciprocating compressor with interstage membrane hydrogen separation and cooling devices; and,
a high pressure storage tank operatively connected to the condensing heat exchanger.

18. The apparatus of claim 15 wherein the apparatus further comprises a gas expansion valve, a heat exchanger and static in line mixers sequentially and operatively connected to the high pressure storage tank.

19. The apparatus of claim 16 wherein the apparatus further comprises a gas expansion valve, a heat exchanger and static in line mixers sequentially and operatively connected to the high pressure storage tank.

20. The apparatus of claim 17 wherein the apparatus further comprises a gas expansion valve, a heat exchanger and static in line mixers sequentially and operatively connected to the high pressure storage tank.

21. The apparatus of claim 15, wherein the apparatus further comprises:
at least two degasification units for the removal of entrained gases from water.

22. The apparatus of claim 16, wherein the apparatus further comprises:
at least two degasification units for the removal of entrained gases from water.

23. The apparatus of claim 17, wherein the apparatus further comprises:
at least two degasification units for the removal of entrained gases from water.
